(19) United States
(12) Patent Application Publication
(10) Pub. No.: US 2016/0281061 A1
(43) Pub. Date: Sep. 29, 2016

(54) TISSUE ARRAY FOR CELL SPHEROIDS AND METHODS OF USE

(71) Applicant: The John Hopkins University, Baltimore, MD (US)

(72) Inventors: Vince Beachley, Baltimore, MD (US); Jennifer H. Elisseeff, Baltimore, MD (US)

(21) Appl. No.: 15/034,410

(22) PCT Filed: Nov. 5, 2014
(86) PCT No.: PCT/US2014/064091

§ 371 Date: May 4, 2016

Related U.S. Application Data
(60) Provisional application No. 61/900,090, filed on Nov. 5, 2013.

(51) Int. Cl. C12N 5/0775; G01N 1/30

(52) U.S. Cl. CPC C12N 5/0667; G01N 1/30; C12N 2533/76

(57) ABSTRACT

The present invention generally features methods of preparing a microarray of cell spheroids, methods of preparing a micromold for embedding spheroids for histology, and methods of screening a library of agents.

### RELATED APPLICATIONS

The present application claims priority to, and the benefit under 35 U.S.C. §119(e) of U.S. provisional patent application No. 61/900,090, entitled “Tissue Array for Cell Spheroids and Methods of Use,” filed Nov. 5, 2013. The entire content of the aforementioned patent application is incorporated herein by this reference.

### BACKGROUND OF THE INVENTION

In vitro cell culture is widely used as a model system to understand cell behavior. However, in vitro conditions are very different from the in vivo environment so it can be difficult to determine the applicability of in vitro observations to whole organisms. The majority of cellular studies are performed on a 2D monolayer culture; however this is not considered the natural environment of cells. 3D cell culture offers a higher degree of biological relevance for in vitro studies. Thus, cells in a 3D microenvironment have shown improved function compared to 2D in vitro. It is hypothesized that differences in cell-cell and cell-matrix adhesion interactions are responsible for the discrepancy between 2D and 3D culture.

A spheroid is a 3D aggregate of living mammalian cells cultured in vitro from tissue explants, established cell cultures or a mixture of both. Cell spheroids can be formed by a variety of methods including hanging drop and seeding on non-adherent substrates. Spheroid research, initially, focused largely on monoculture of cells as 3D aggregates. However, heterologous spheroids with more than one cell type have been used to investigate the interactions of different cell types in both normal tissue and tumor development. Currently cell spheroids are cultured to study the behavior of many different cellular systems, such as cancer cells and stem cells, and to do preliminary testing of new drugs or other therapeutics. The internal environment of a spheroid is dictated by the metabolism and adaptive responses of cells with a well-defined morphological and physiological geometry. Beyond a critical size (>500 uM) most monotypic spheroids develop concentric layers of heterogeneous cell populations with proliferating cells at the periphery and a layer of quiescent cells close to the necrotic core. This heterogeneous arrangement of cells in a spheroid mimics initial avascular stages of early tumors. Another type of monotypic spheroid forms well organized acini-like structures with a central lumen when epithelial cells are cultured over reconstituted basement membrane. These monotypic spheroids are able to mimic important in vivo morphology, although much of the biological complexity is lost. Because of their superior replication of the natural cellular environment, spheroids have been extensively used as tools for mechanistic assays and for probing cell-cell interactions. One application is the use of spheroids to investigate mechanisms of tumor biology. Chemotherapeutic drugs are also tested on multicellular spheroids because cells in this microenvironment exhibit great resistance than the same cell type in 2D culture. Liver cell spheroids are commonly used for drug toxicity screening and several companies offer liver micro tissue drug screening services.

Currently several products are being marketed for high throughput culture of cell spheroids to expand the usefulness of this promising technology. However, high throughput technologies for analyzing spheroids are limited. Spectrophotometric assays can be used efficiently with high throughput culture systems, but the information that results from these tests is limited compared to conventional histological analysis. Histological analysis with embedding and sectioning of spheroids is difficult and time consuming.

Accordingly, there is a need in the art for improved methods for embedding, sectioning and staining spheroids simultaneously in large quantities.

### SUMMARY OF THE INVENTION

Histological analysis of cell spheroids is very time consuming if each spheroid is embedded, sectioned, and stained individually. Sectioning and staining several spheroids together is difficult and it becomes especially tricky to keep samples from different groups separated. Described herein is a simple method of embedding spheroid in a microarray. The core advantage of this system is that the specific location of each sample is easily recorded so that a large number of unique samples (e.g. 40 or more) can be embedded in one block. Further, these samples are maintained on the same plane so it is possible to cut single sections that contain each of the samples and stain and analyze them in a single slide. The diameter of spheroids is commonly hundreds of microns, so a great number of sections can be cut for each slide allowing many different types of analysis. This system is an excellent complement to current advances in scaling up spheroid production in 96 and 384 well plates.

Accordingly, in a first aspect, the invention provides a method for preparing a microarray of cell spheroids that involves culturing a plurality of cell spheroids in at least one array plate having a top surface and a bottom surface and a plurality of holes in the plate, where the plate is configured to accommodate a plurality of hanging drops, where the hanging drops harbor one or more spheroids, preparing a micromold having an array of wells, transferring the cell spheroids to the micromold wells, and filling the micromold with agarose.

Optionally, the micromold is a single piece micromold. Alternatively, the micromold is comprised of multiple pieces. In one embodiment, the micromold is made of plastic or silicone. In a related embodiment, the micromold is made of polydimethylsiloxane (PDMS).

In another embodiment, the method further includes a step of placing a mounting block over the micromold before filling the micromold with agarose.

In one aspect, the invention features a method of preparing a microarray of cell spheroids comprising culturing a plurality of cell spheroids in at least one array plate comprising a top surface and a bottom surface and a plurality of holes therein, and configured to accommodate a plurality of hanging drops, wherein the hanging drops comprise one or more spheroids, preparing a micromold having an array of through-holes or wells, transferring the cell spheroids to the micromold, placing a mounting block over the micromold; and filling the micromold with agarose.

In another aspect, the invention features a method of preparing a micromold of embedded spheroids for histology comprising culturing a plurality of cell spheroids in at least one array plate comprising a top surface and a bottom surface and a plurality of holes therein, and configured to accommodate a plurality of hanging drops, wherein the hanging drops comprise one or more spheroids, preparing a micromold by pressing it against a hydrophobic surface, transferring the cell spheroids to the micromold, placing a mounting block over the micromold, filling the micromold with agarose, and embedding the micromold in paraffin or cryomount.

In one embodiment, each drop hangs from a corresponding one of the plurality of said holes and extends beneath the hole, wherein the number of hanging drops that the array plate can accommodate is equal to or less than the number of holes in the at least one array plate.

In another embodiment, the methods of the above aspects further comprises embedding the micromold in paraffin or cryomount.

In another embodiment, the hydrophobic surface is a silicone substrate.

In another embodiment of the above aspects, the method further comprises sectioning the micromold and transferring the sections to slides.

In another further embodiment, the method further comprises staining the slides.

In one embodiment, the cell spheroids are derived from healthy subjects or subjects with diseases selected from the group consisting of degenerative diseases, cancer diseases, autoimmune and/or inflammatory diseases, cardiovascular diseases and neurological disorders. In a further embodiment, the cell spheroids are derived from stem cells. In another further embodiment, the spheroid is used to model a disease or disorder.

In another embodiment of the present invention, the cell spheroids are treated with an agent during culturing in the at least one array plate.

In another aspect, the invention features a method of screening a library of agents comprising culturing a plurality of cell spheroids in at least one array plate comprising a top surface and a bottom surface and a plurality of holes therein, and configured to accommodate a plurality of hanging drops, wherein each hanging drop comprises one or more spheroids, introducing an agent or a combination of agents into each hanging drop, preparing a micromold having an array of through-holes or wells, transferring the cell spheroids to the micromold, placing a mounting block over the micromold, filling the micromold with agarose; and embedding the micromold in paraffin or cryomount.

In certain embodiments, the step of preparing the micromold involves pressing the micromold against a hydrophobic surface. In one embodiment, the method further comprises sectioning the micromold and transferring the sections to slides. In a further embodiment, the method comprises staining the slides for a marker of interest. In exemplary embodiments, the marker is a protein.

In another embodiment, the one or more separate hanging drop is treated with the same agent or with a different concentration of the same agent. In a related embodiment, the one or more separate hanging drop is treated with a different agent or a different concentration of the different agent. In still another embodiment, the one or more hanging drops are treated as controls.

In another embodiment, the agent is selected from one or more of the group consisting of native or endogenous ligand or ligands, a combinatorial library of small molecules, hormones, antibodies, polysaccharides, anti-cancer agents, natural products, terrestrial products, marine natural products, a molecule that binds with high affinity to a biopolymer such as a protein, a nucleic acid, and a polysaccharide, a purified or isolated biological molecule such as a protein, a nucleic acid, a silencing RNA (siRNA), a micro RNA (miRNA), and a short hairpin RNA (shRNA).

In further embodiments, detection of the marker indicates activity of the agent. In other further embodiments, absence of the marker indicates activity of the agent.

In certain embodiments, the method of the aspects described herein is an in vitro method.

The invention also features a micromold for embedding spheroids comprising a plurality of cell spheroids and a mounting block, wherein the micromold is filled with agarose. In one embodiment, the micromold is embedded in paraffin or cryomount.

A further aspect of the invention provides an agarose-embedded array that contains spheroids within array elements. In one embodiment, multiple array elements contain one or more spheroids. In another embodiment, each array element contains one or more spheroids.

Another aspect of the invention provides a method for comparing the staining intensities of different spheroids without normalizing observed staining intensity values to an external value, the method involving staining an agarose-embedded array of the invention (optionally, one that has been fixed and/or sectioned), imaging the array on a single slide to obtain staining intensity values of different spheroids of the array, and directly comparing the staining intensity values of the different spheroids of the array, in the absence of normalization to an external value or control.

Definitions

The following terms are provided solely to aid in the understanding of this invention.

These definitions should not be construed to have a scope less than would be understood by a person of ordinary skill in the art.

In this disclosure, “comprises,” “comprising,” “containing” and “having” and the like can have the meaning ascribed to them in U.S. Patent law and can mean “includes,” “including,” and the like; “consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used in this specification and the appended claims, the singular forms “a,” “an” and “the” include plural references unless the content clearly dictates otherwise.

The term “agent “is meant to refer to any chemical entity, pharmaceutical, drug, and the like that is a candidate for use to treat or prevent a disease, illness, sickness, or disorder of bodily function. Agents comprise both known and potential therapeutic compounds. A test agent may be determined to be therapeutic by screening using the screening methods, devices, and/or systems of the present disclosure. In certain embodiments of the present disclosure, test agents may include native or endogenous ligand or ligands, a combinatorial library of small molecules, hormones, antibodies, polysaccharides, anti-cancer agents, natural products, terrestrial products, marine natural products, a molecule that binds with high affinity to a biopolymer such as a protein, a nucleic acid, and a polysaccharide, a purified or isolated biological molecule such as a protein, a nucleic acid, a silencing RNA (siRNA), a micro RNA (miRNA), and a short hairpin RNA (shRNA).

As used herein, the term “cell” refers to any eukaryotic or prokaryotic cells (e.g., bacterial cells such as E. coli, yeast cells, mammalian cells, avian cells, amphibian cells, plant cells, fish cells, and insect cells), whether located in vitro or in vivo or combinations thereof. The term “cell” also refers to aqueous fluids or solutions containing one or more cells in a suspension or in clusters or aggregates.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), other cell population maintained in vitro, or combinations thereof.

As used herein, the term “spheroid” refers to an aggregate, cluster or assembly of cells cultured to allow three-dimensional growth in contrast to the two-dimensional growth of cells in either a monolayer or cell suspension (cultured under conditions wherein the potential for cells to aggregate is limited). The aggregate may be highly organized with a well-defined morphology or it may be a mass of cells that have clustered or adhered together with little organization reflecting the tissue of origin. It may comprise a single cell type (homotypic) or more than one cell type (heterotypic). Optionally, the cells are primary isolates, but in certain embodiments, they may also include a combination of primary isolates with an established cell line(s). Particular cell ‘types’ include somatic cells, stem cells, progenitor cells and cancer stem cells.

### BRIEF DESCRIPTION OF THE DRAWINGS

FIG. 1 shows steps in the method of preparing a tissue microarray for cell spheroids.

FIG. 2 shows spheroids stained with Alizarin red.

FIGS. 3A to 3C show a single piece mold made of PDMS, viewed from various angles.

### DETAILED DESCRIPTION OF THE INVENTION

The present invention provides, generally, a simple and efficient method that allows spheroids to be embedded and sectioned, and stained simultaneously in large quantities.

### Spheroids and Production

Spheroids are spherical clusters of cell colonies that may be formed by self-assembly when cell-cell interactions dominate over cell-substrate interactions. Spheroids may generally be defined as clusters or aggregates of cells and/or cell colonies that may be formed by self-assembly when cell-cell interactions dominate over cell-substrate interactions.

Spheroids may be formed from various cell types, for example, primary cells, cell lines, tumor cells, stem cells, etc. Spheroids may have spherical or irregular shapes. Spheroids may contain heterogeneous populations of cells, cell types, cells of different states, such as proliferating cells, quiescent cells, and necrotic cells. Spheroids may mimic tumors and may serve as excellent physiologic tumor models known to provide more reliable and meaningful therapeutic readouts. Spheroids may produce results and/or measurements that are consistent and/or reproducible. A three-dimensional cell culture preparation method is disclosed in WO 2004/101743 A2 and WO 2005/095585 A1, incorporated by reference in its entirety herein.

An exemplary method for the formation of hanging drops is the following, described by Foty et al. (J Vis Exp. 2011 May 6; (51). pii: 2720; incorporated by reference in its entirety herein).

### Preparation of a Single Cell Suspension

1. Adherent cell cultures should be grown to 90% confluence, whereupon monolayers should be rinsed twice with PBS. After draining well, add 2 mls (for 100 mm plates) of 0.05% trypsin-1 mM EDTA, and incubate at 37° C. until cells detach. Stop trypsinization by adding 2 mls of complete medium and gently use a 5 ml pipette to triturate the mixture until cells are in suspension. Transfer cells to a 15 ml conical tube.
2. Add 40 μl of a 10 mg/ml DNAse stock and incubate for 5 minutes at RT. Vortex briefly and centrifuge at 200×G for 5 minutes.
3. Discard supernatant and wash pellet with 1 ml complete tissue culture medium. Repeat, then resuspend cells in 2 mls of complete tissue culture medium.
4. Count the cells using a hemacytometer, or automated cell counter and adjust concentration to 2.5×10⁶ cells/ml. For this demonstration a BioRad TC10 automated cell counter was used.

### Formation of Hanging Drops

5. Remove the lid from a 60 mm tissue culture dish and place 5 mls of PBS in the bottom of the dish. This will act as a hydration chamber.
6. Invert the lid and use a 20 μl pipettor to deposit 10 μl drops onto the bottom of the lid. Make sure that drops are placed sufficiently apart so as to not touch. It is possible to place at least 20 drops per dish.
7. Invert the lid onto the PBS-filled bottom chamber and incubate at 37° C./5% CO₂/95% humidity, monitor the drops daily and incubate until either cell sheets or aggregates have formed. A stereo microscope can be used to assess aggregate formation.
8. Once sheets form, they can be transferred to round-bottom glass shaker flasks containing 3 mls of complete medium and incubated in a shaking water bath at 37° C. and 5% CO₂ until spheroids form.

Hanging drop array systems allow for efficient formation of uniformly-sized spheroids and/or long-term spheroid cultures in a standardized plate format compatible with various commercially available high throughput (HTS) systems, which make these systems ideal for commercialization for wider use. The hanging drops of fluid may contain one or more of the following: suspension and/or aggregates of cells. In certain embodiments, the hanging drops contain physical, chemical, biological entities, or combinations thereof. The hanging drop assay can also be modified to include more than one cell type.

Hanging drop plates are commercially available from a number of resources. For example, 3D Biomatrix provides 96 well and 384 well hanging drop plates. An exemplary protocol for culture of spheroids in hanging drops is as follows:
1. Add water or buffer to the reservoirs located on the peripheral rim of the plate and tray which are divided by baffles into sections. Add 2 mL per plate reservoir section and 1 mL per tray reservoir section. Note: if liquid is used to fill the reservoirs, tilting the plate may result in spilling of the liquid and contamination of hanging drops. Optional: Prepare 6 mL per plate 0.5-1.0% agarose solution in water or buffer, heat to melt the agarose, and allow the solution to cool to ˜50° C. Add pre-heated agarose to reservoirs as described above for buffer or water.
2. Prepare cell suspension to the desired concentration. Each hanging drop holds 20 to 30 μL, so prepare accordingly-e.g. if 2500 cells/25 μL drop is desired, dilute cell suspension to 100 cells/μL.
3. Form hanging drops by pipetting 20 to 30 μL of cell suspension to each well from the top side of the plate. Hanging drops should be formed on and confined to the bottom of the plate.
4. Put the lid on and place the assembly into a cell culture incubator. Within hours, individual cells should start to aggregate and eventually form into spheroids. Spheroid formation time varies with cell types.

It is known to one skilled in the art that there are many different ways to make spheroids, and any known method is contemplated for use in the present invention. For example, Fennema et al. (Trends in Biotechnology, February 2013. Vol.31, no. 2, incorporated by reference in its entirety herein) teaches methods of 3D culture of spheroids.

In certain embodiments, one spheroid forms per well, and the spheroid diameter is controlled by the cell type and number of cells added to each well.

The methods and/or systems of the present invention provide the ability to grow cells of uniform and adjustable cellular aggregate size (e.g., size/volume of cellular aggregate may be control by geometry of plate structure, cell seeding number, or culture time) and are suitable for high-throughput screening. High-throughput screening (HTS), generally means that the embodiment is compatible with microscopy, analytical, and/or automated systems that are used in drug discovery and relevant fields of chemistry and biology. For example, HTS allows researchers to perform large number of tests, for example 100 to 100,000 tests, in a day. In certain embodiments, the number of tests that can be performed may be 100 to 10,000, 500 to 10,000, 100 to 20,000, 1000 to 30,000, 1000 to 50,000, 10,000 to 80,000, etc. HTS allows researchers to identify chemical and biological entities of relevance and understand biological processes. Mainstream HTS instruments are designed to perform operations or tasks, such as liquid handling, imaging, microscopy, or optical detection, on samples contained on a microtiter plate that complies with ANSI/SBS standards. In some embodiments, the device (array plate or combination of array plate with lid and bottom plate) complies with standards, for example present ANSI/SBS standards, therefore allowing the device to be used with HTS instruments, which means the generation and assessment of hanging drops or spheroids can be easily scaled up.

As discussed herein, certain embodiments provide a multiplex (e.g., 1536, 384, 96, etc.) hanging drop array plate that provides easy handling and media exchange procedures. In other embodiments, the access holes are arranged in other suitable multiplex configurations, in row and columns, such as 18 (3 by 6), 25 (5 by 5), 72 (6 by 12), 100 (10 by 10), or 625 (25 by 26) holes. The use of standardized (e.g., 16 by 24 384-well, 8 by 12 96-well) formats that comply with standards, for example present standards set by ANSI/SBS (American National Standards Institute/Society of Biomolecular Sciences), offers compatibility with most commercially available HTS instruments. The hanging drop array plates described herein find use, for example, in preparing a micromold of embedded spheroids for histology, and for use as a high-throughput 3D screening/testing platform for a variety of applications.

Certain embodiments may be suitable for mass production of cellular aggregates. In some embodiments, each device allows the formation of 384 spheroids in hanging drops. By using automated systems and a plurality of devices, one can form, for example, 1,000 to 100,000 hanging drops, each containing cells that will form spheroids, within a reasonable period of time, for example within 5 minutes, 15 minutes, 1 hour, 2 hours, 5 hours, 10 hours, or 24 hours.

Certain embodiments are suitability for long-term culture of cellular aggregates prior to embedding in a tissue microarray mold. For example, in certain embodiments cellular aggregates may be cultured for at least 1, 2, 3, 4, 5 or 6 weeks. For example, in certain embodiments cellular aggregates may be cultured for between 1 to 6 weeks, 1 to 2 weeks, 1 to 4 weeks or 2 to 5 weeks.

Certain embodiments are suitability for culture of cellular aggregates for shorter periods of time prior to embedding in a tissue microarray mold. For example, in certain embodiments cellular aggregates may be cultured for at least 30 minutes, 1 hour, 2 hours, 3 hours, 5 hours, 8 hours, 12 hours 24 hours, 2 days, 3 days, or 6 days. For example, in certain embodiments cellular aggregates may be cultured for up to 30 minutes, 1 hour, 2 hours, 3 hours, 5 hours, 8 hours, 12 hours 24 hours, 2 days, 3 days, 6 days or 7 days. For example, in certain embodiments cellular aggregates may be cultured for between 30 minutes to 7 days, 2 hours to 24 hours, 30 minutes to 48 hours, 1 hour to 5 days, or 1 hour to 7 days.

In order to culture spheroids over various periods of time including a long period of time, the osmolality of the cell culture media in the hanging drops is kept in certain embodiments within a relatively stable range. In certain embodiments, a relatively stable range may be maintaining the desired parameters of the hanging drops to ±1%, ±3%, ±5%, ±8%, ±10%, ±15%, ±20%, or ±25% of the desired or stated parameters. In certain embodiments, a relatively stable range may be maintaining the desired or stated parameters of the hanging drops to a sufficient range of variation such that the end results of the culturing may be achieved or substantially achieved. In certain embodiments, the osmolality of the cell culture media in the hanging drops is kept within a relatively stable range. For example, within 10% to 20% of the initial osmolality measurements. In other examples, within 3% to 20%, 5% to 15%, 5% to 25%, 5% to 10%, or 15% to 20% of the initial osmolality measurements. In certain embodiments, culture of spheroids can be kept in a stable range for 1 to 6 weeks. For example, in certain embodiments culture of spheroids can be kept in a stable range for at least 30 minutes, 1 hour, 2 hours, 3 hours, 5 hours, 8 hours, 12 hours 24 hours, 2 days, 3 days, or 6 days. For example, in certain embodiments culture of spheroids can be kept in a stable range for between 30 minutes to 7 days, 2 hours to 24 hours, 30 minutes to 48 hours, 1 hour to 5 days, or 1 hour to 7 days. Other ranges are also contemplated.

Encompassed by the present invention is the ability to generate highly reproducible spheroid formation(s) in the hanging drops. Because spheroids can be formed with substantially the same initial number of cells, and the spheroids are formed in isolated volumes, the growth of spheroids are highly reproducible, and fusing of neighboring spheroids, which produces variation in size, is avoided since contact between individual spheroids is avoided. In certain embodiments, the variation in size between spheroids can be maintained within 3% to 5% throughout the culture period. In certain embodiments, the variation in size between spheroids can be maintained within 3% to 5%, 2% to 6%, 1% to 6%, or 3% to 6% throughout the culture period. In certain embodiments, the variation in size between spheroids can be maintained within 3% to 5%, 2% to 6%, 1% to 6%, or 3% to 6% throughout a substantial portion of the culture period.

Spheroids can be prepared from a number of cells. In certain embodiments, the cell spheroids are derived from healthy subjects or subjects with diseases selected from the group consisting of degenerative diseases, cancer diseases, autoimmune and/or inflammatory diseases, cardiovascular diseases and neurological disorders. The spheroids can be used to model a disease or disorder.

Spheroids can in principle be produced from any desired tissue or organ from any animal by disrupting a sample of the tissue or organ, optionally disrupting to individual cells or to small groups of cells. For example, the tissue which may be used for spheroid preparation may be a normal or healthy biological tissue, or may be a biological tissue afflicted with a disease or illness, such as a tissue or fluid derived from a tumor. In certain embodiments, the tissue is a mammalian tissue. Also encompassed are metastatic cells. The tissue may be obtained from a human, for example from a patient during a clinical surgery or from biopsies. The tissue may also be obtained from animals such as mice, rats, rabbits, and the like. It is also possible according to the invention to prepare spheroids from stem cells, progenitor cells or cancer stem cells.

Besides cells originating from tumor tissue, other cells with various indications such as smooth muscle cells, adipocytes, neural cells, stem cells, islet cells, foam cells, fibroblasts, hepatocytes and bone marrow cells, cardiomyocytes and enterocytes are also encompassed within the present invention.

Also within the scope of the present invention is the possibility to rebuild a metastatic microtumor e.g., tumor cells with hepatocytes, or tumor cells with bone marrow cells. Also useful within the invention are primary cancer cells such as gastric, colon and breast primary cancer cells and metastatic cells. Also encompassed by the invention are primary normal (healthy) cells such as endothelial cells, fibroblasts, liver cells, and bone marrow cells.

Optionally, the cells are directly derived from the tissue of a patient or healthy donor, a tissue derived from a biopsy, surgical specimens, an aspiration or a drainage and also cells from cell-containing bodily fluids.

Cells from cell lines may also be used. These may be initially cultured as a monolayer to generate more cells; trypsinization may be used for cell dissociation of a monolayer cell culture. In certain embodiments, spheroids can be prepared from cells from a tissue or an organ of a subject, for example healthy subjects or subjects with diseases selected from the group consisting of degenerative diseases, cancer diseases, autoimmune and/or inflammatory diseases, cardiovascular diseases and neurological disorders. In certain embodiments, the cell spheroids are derived from stem cells.

The multicellular spheroids according to the invention can also be characterized in that they exhibit characteristics that substantially mimic those of the tissue of origin, such as: antigen profile and/or genetic profile, tumor biologic characteristics, tumor architecture, cell proliferation rate(s), tumor microenvironments, therapeutic resistance and composition of cell types. Optionally, they exhibit an antigen profile and genetic profile which is substantially identical to that of the tissue of origin.

Thus, the spheroids of the invention exhibit a substantially similar/identical behavior to that of natural cell systems, e.g., with respect to organization, growth, viability, cell survival, cell death, metabolic and mitochondrial status, oxidative stress and radiation response as well as drug response.

### Methods

The present invention features in certain aspects methods of preparing a microarray of cell spheroids comprising culturing a plurality of cell spheroids in at least one array plate comprising a top surface and a bottom surface and a plurality of holes therein, and configured to accommodate a plurality of hanging drops, wherein the hanging drops comprise one or more spheroids, preparing a micromold by pressing it against a hydrophobic surface, transferring the cell spheroids to the micromold, placing a mounting block over the micromold, and filling the micromold with agarose.

The present invention also features a method of preparing a micromold for embedding spheroids for histology comprising culturing a plurality of cell spheroids in at least one array plate comprising a top surface and a bottom surface and a plurality of holes therein, and configured to accommodate a plurality of hanging drops, wherein the hanging drops comprise one or more spheroids, preparing a micromold by pressing it against a hydrophobic surface, transferring the cell spheroids to the micromold, placing a mounting block over the micromold, filling the micromold with agarose and embedding the micromold in paraffin or cryomount.

In certain embodiments, the hydrophobic surface is a silicone substrate. Silicones are inert, synthetic compounds with a variety of forms and uses, and are typically heat-resistant and rubber-like. Silicones are polymers that include silicon together with carbon, hydrogen, oxygen, and sometimes other elements. In some embodiments, the silicone substrate is polydimethylsiloxane (PDMS). Polydimethylsiloxane (PDMS) belongs to a group of polymeric organosilicon compounds that are commonly referred to as silicones. PDMS is the most widely used silicon-based organic polymer, and is particularly known for its unusual rheological (or flow) properties. PDMS is optically clear, and, in general, inert, non-toxic, and non-flammable. It is also called dimethicone and is one of several types of silicone oil (polymerized siloxane). It is understood that the material of the mold is not limited to any particular material. In certain embodiments, the mold is optionally comprised of PDMS and silicone.

Optionally, the bottom of the mold is a separate material and is held together with external pressure. Accordingly, in certain embodiments, the mold can be two pieces. In other embodiments, the mold can be one piece.

The present invention advantageously provides that spheroids are maintained in separate compartments to allow for sample identification. Moreover, spheroids are on the same plane so can be sectioned and stained on one slide.

### Histology

Histology sample preparation prepares tissue specimens for sectioning, staining and diagnosis. The standard paraffin process (tissue processing) moves specimens through a series of steps so the soft tissue is supported in a medium that allows sectioning.

The methods of the present invention as described herein further comprise embedding the micromold in paraffin or cryomount. The micromold can be sectioned and transferred to slides for staining.

The standard steps are: fixation that preserves the tissue, processing that dehydrates, clears and infiltrates the tissue with paraffin wax, embedding that allows orientation of the specimen in a “block” that can be sectioned and is easy to store and handle, and sectioning using a microtome to produce very thin sections that are placed on a microscope slide ready for staining. Frozen sectioning is an alternative preparation technique that quickly freezes tissue to preserve it and provide sufficient hardness so it can be sectioned immediately using a cryostat.

One advantage of the present invention is that the spheroids are on the same plane, and so they can be sectioned and stained on one slide. Accordingly, the simple and efficient methods of the present invention allow spheroids to be embedded and sectioned, and stained simultaneously in large quantities.

Indeed, because the current invention provides for staining and imaging of spheroids in the same batch, on the same slide, where cells/spheroids are, e.g., a component of a spheroid array, quantitative comparison of staining intensities between cells or spheroids is possible, even in the absence of normalization (e.g., without need to normalize staining intensities obtained for individual spheroids to an external value, instead performing a direct comparison of raw intensity values between array elements (spheroids).

Microarray blocks are sectioned with a microtome or cryostat where the block is held at a precise angle at its base and a thin slice or section (˜5-20 um) is cut from the top surface of the block with a razor blade. The thin sections are then transferred to a microscope slide where they can be stained to reveal images or identify biochemical composition of each individual spheroid in the array. It is not difficult to precisely line up the spheroid array so all of the included spheroids are cut in the same section. Staining all of the spheroids on one slide saves time and money and allows the researcher to conduct a larger quantity of tests on each sample.

### Diagnostic and Screening Applications

Since the multicellular spheroids according to the invention are substantially identical to in vivo cell systems, these spheroids can thus be used for diagnostic and/or therapeutic purposes, for example, pharmacokinetic profiling, pharmacodynamic profiling, efficacy studies, cytotoxicity studies, penetration studies of compounds, therapeutic resistance studies, antibody generation, personalized or tailored therapies, RNA/DNA ‘drug’ testing, small molecule identification and/or testing, biomarker identification, tumor profiling, hyperthermia studies, radioresistance studies and the like.

In the methods of the invention, the cell spheroids may be treated with one or more agents during culturing.

For example, the cell spheroids can be obtained from benign or malignant tissues or from primary cells and used for the screening of agents or compounds, for example, as new therapeutic agents or screening for agents, e.g. chemotherapeutics wherein the response of the spheroid to the agent can be determined It is thus possible to see whether an agent has an effect and/or side effects on the multicellular spheroid, e.g., whether it causes cell death (apoptosis) or other biologic effect.

In one aspect, the invention features a method of screening a library of agents comprising culturing a plurality of cell spheroids in at least one array plate comprising a top surface and a bottom surface and a plurality of holes therein, and configured to accommodate a plurality of hanging drops, wherein each hanging drop comprises one or more spheroids, introducing an agent or a combination of agents into each hanging drop, preparing a micromold by pressing it against a hydrophobic surface, transferring the cell spheroids to the micromold, placing a mounting block over the micromold, filling the micromold with agarose, and embedding the micromold in paraffin or cryomount.

In embodiments, of the invention, one or more separate hanging drops is each treated with the same agent or each is treated with a different concentration of the same agent. In other embodiments, one or more separate hanging drops is each treated with a different agent or each is treated with a different concentration of the different agent. Further, one or more hanging drops are treated as controls.

The agent is not limited, and can be any agent. For example, the agent can be selected from one or more of the group consisting of: native or endogenous ligand or ligands, a combinatorial library of small molecules, hormones, antibodies, polysaccharides, chemotherapeutic agents, natural products, terrestrial products, marine natural products, a molecule that binds with high affinity to a biopolymer such as a protein, a nucleic acid, and a polysaccharide, a purified or isolated biological molecule such as a protein, a nucleic acid, a silencing RNA (siRNA), a micro RNA (miRNA), and a short hairpin RNA (shRNA). chemotherapeutic agents may include: alkylating agents, antimetabolites, anthracyclines, plant alkaloids, topoisomerase inhibitors, and other anti-tumor agents, antibodies such as monoclonal, single chain or fragments thereof and the new tyrosine kinase inhibitors e.g., imatinib mesylate, small molecules, tyrosine kinase receptor inhibitors, anticalins, aptamers, peptides, scaffolds, biosimilars, and generic drugs.

Optionally, the method further comprises sectioning the micromold and transferring the sections to slides, and staining the slides for a marker of interest. The marker is not to be limited to any marker in particular. For example, the marker can be a protein.

In certain examples, detection of the marker indicates activity of the agent. In other examples, absence of the marker indicates activity of the agent. For example, detection of the marker is compared to a control. The marker may show 1-fold, 2-fold, 3-fold, 4-fold, 5-fold, 6-fold, 7-fold, 8-fold, 9-fold or more increase in the treated droplets as compared to the control. In other examples, the marker is expected to be present in the control, and treatment of the droplets with the agent of interest may result in a decrease of the marker, for example a 1-fold, 2-fold, 3-fold, 4-fold, 5-fold, 6-fold, 7-fold, 8-fold, 9-fold or more increase in the treated droplets as compared to the control.

### Spheroids as a Model for Cancer

There is a developing body of literature describing the use of spheroids as in vitro tumor models. Both monotypic and heterotypic spheroids have proven useful as tumor models. Heterotypic spheroids offer the ability to investigate interactions between different cell types in the tumor microenvironment. Monotypic spheroids comprised of malignant cells offer the advantage of simplicity and they can effectively represent initial avascular stages of early tumors. Accordingly, spheroids can be prepared according to the methods of the present invention and used as in vitro tumor models.

### Embryoid Bodies

Another potential application of the presently claimed methods of preparing spheroids and spheroid microarrays is to embryoid bodies, an in vitro model of mouse embryogenesis. Embryoid bodies (EBs) are three-dimensional aggregates of pluripotent stem cells. The pluripotent cell types that comprise embryoid bodies include embryonic stem cells (ESCs) derived from the blastocyst stage of embryos from mouse (mESC), primate, and human (hESC) sources. Additionally, EBs can be formed from embryonic stem cells derived through alternative techniques, including somatic cell nuclear transfer or the reprogramming of somatic cells to yield induced pluripotent stem cells (iPS). Similar to ESCs cultured in monolayer formats, ESCs within embryoid bodies undergo differentiation and cell specification along the three germ lineages—endoderm, ectoderm, and mesoderm—which comprise all somatic cell types.

In contrast to monolayer cultures, however, the spheroid structures that are formed when ESCs aggregate enables the non-adherent culture of EBs in suspension, making EB cultures inherently scalable, which is useful for bioprocessing approaches, whereby large yields of cells can be produced for potential clinical applications. Additionally, although EBs largely exhibit heterogeneous patterns of differentiated cell types, ESCs are capable of responding to similar cues that direct embryonic development. Therefore, the three-dimensional structure, including the establishment of complex cell adhesions and paracrine signaling within the EB microenvironment, enables differentiation and morphogenesis which yields microtissues that are similar to native tissue structures. Such microtissues are promising to directly or indirectly repair damaged or diseased tissue in regenerative medicine applications, as well as for in vitro testing in the pharmaceutical industry and as a model of embryonic development. See, for example, Desbaillets et al. (Experimental Physiology (2000) 85.645-651, incorporated by reference in its entirety herein).

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

### EXAMPLES

### Example 1

### Method of Preparing a Microarray of Cell Spheroids.

Histological analysis of cell spheroids is very time consuming if each spheroid is embedded, sectioned, and stained individually. Sectioning and staining several spheroids together is difficult and it becomes especially tricky to keep samples from different groups separated. Here a simple method of sorting and embedding spheroids is presented. This method makes it easy to section many (prototype up to 40) spheroids in the same block and on the same plane, while maintaining the location of each sample. This system is an excellent complement to current advances in scaling up spheroid production in 96 and 384 well plates.

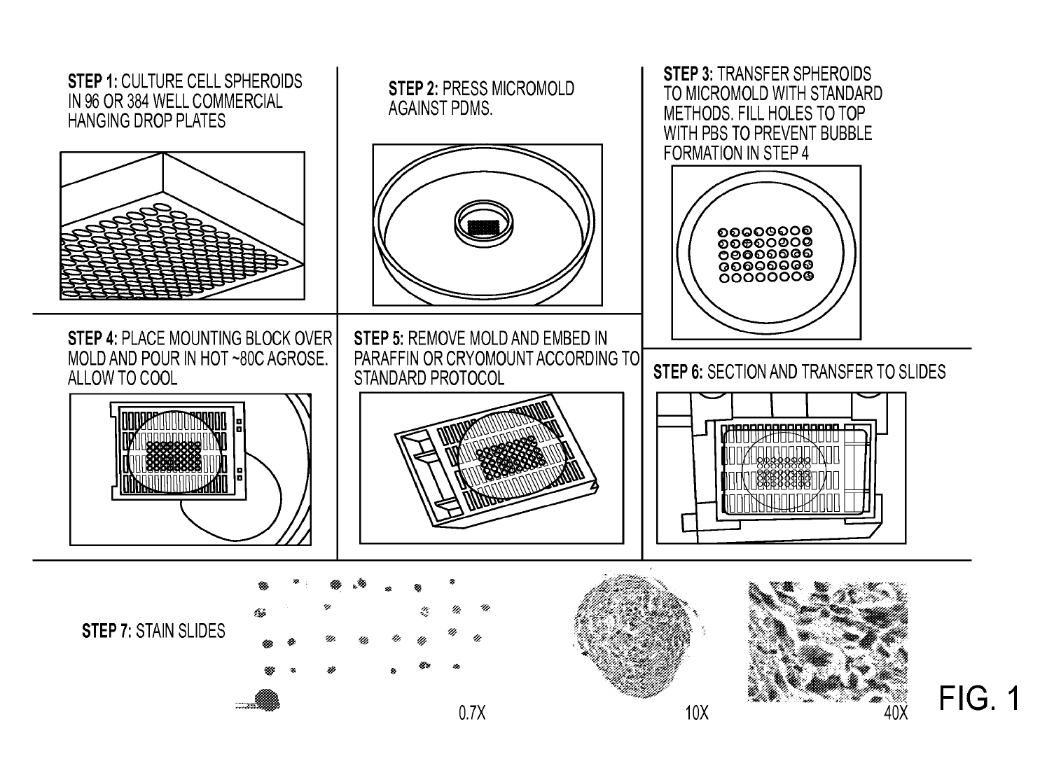

As a first step, cell spheroids are cultured in in 96 or 384 well commercial hanging drop plates. Next, a micromold is pressed against PDMS. The spheroids are then transferred to the micromold with standard methods, for example with a pipette. Holes are filled to the top with PBS to prevent bubble formation. A mounting block is then placed over the mold and agarose (˜80 C) is poured into the mold and allowed to cool for a period of time. The mold is removed and embedded in paraffin or cryomount according to standard protocol. The mold is sectioned and transferred to slides according to standard protocol, and staining is performed on the slide. FIG. 1 shows an outline of this procedure.

### Methods

At time of harvest spheroids were washed with PBS and fixed for 1 hr in 4% paraformaldehyde in a 96 well plate. Spheroids were washed with distilled water and pipetted into the chambers of a plastic mold pressed against a PDMS backing. A piece of fresh tissue was put into one corner of the array to mark orientation and the placement of each spheroid was recorded. The mold was infiltrated with a 2% agarose solution in water at 80 C and allowed to cool and gel. The agarose block was removed and dehydrated and infiltrated in paraffin similar to previously described. Blocks were immersed in graded ethanol solutions (100 ml, 30%, 50%, 70%, 80%, 95%×2, 100%×2) for 3 hrs each and 100% again overnight. Ethanol solutions were cleared with HistoClear II (100 ml) 3 times for 2 hours and once overnight and infiltrated with paraffin (100 ml, 60 C, 4×2 hrs) and cast in paraffin. Paraffin blocks were sectioned at 5 μm. Sections were stained with H&E, masson's trichrome to assess cell/ECM organization and collagen content. Calcium was stained with alizarin red for 5 minutes followed by brief rinsing in acetone, acetone:xylene, and xylene. Slides were imaged at 20× with a slide scanner. Methods of parrafin infiltration into agarose are known in the art, for example as described in Yan et al. (J Histochem Cytochem (2007) 55, 21; incorporated by reference in its entirety herein).

### Embedding Spheroids in Agarose

A detailed method was experimentally identified and used to achieve enhanced embedding of a spheroid array. Without wishing to be bound by theory, the present process was believed to function by reducing air bubble formation, which has been the main reason that failure of the current process can sometimes occur. The process arrived at for embedding spheroids in agarose was the following:
a.) The entire mold was filled with water.
b.) A pipette tip was immersed under the water surface and used to squirt water into the small wells of the mold array (e.g., the wells of a 40 well micromold) to remove air bubbles.
c.) Water was aspirated from the large cavity of the mold, leaving water only in the small cavities of the mold array (e.g., 40 small cavities).
d.) Individual spheroids were sucked into a pipette tip with 5-100 uL of water (10 uL, 100 uL, or 200 uL tip).
e.) The spheroid was allowed to fall to the very outlet (opening at tip) of the pipette tip.
f.) The pipette tip was touched to one of the water filled cavities of the mold (e.g., a 40 well array of the mold) and was held in contact for several seconds.
g.) The spheroid was thereby allowed to fall to the bottom of the water filled cavity.
h.) Steps (c)-(g) were repeated until all desired wells contained a spheroid.
i.) All desired small wells of the mold (e.g., 40 small wells arrayed in a micromold) were now filled with water and had a spheroid (or spheroids) resting on the bottom. The larger top cavity of the mold was empty.
j.) Liquid agarose (at ˜70° C.) was added to the top cavity.
k.) A plastic tissue cassette was placed flat on the mold and agarose was pipetted over it so that the mold was embedded in the agarose.
l.) The liquid agarose diffused into the water/spheroid filled wells, such that the wells also contained liquid agarose solution that gelatinized and encapsulated the spheroids.
m.) After agarose gelatinization, the tissue cassette was lifted vertically from the mold, thereby removing the agarose containing the embedded spheroid microarray from the mold.

### Experiment 1

An experiment was conducted to study the effect of tissue particles on adiopose derived stem cell differentiation in cell/particle spheroids. There were 8 different groups (particle types) and each group was incubated in one of 4 different types of differentiation induction media. To illustrate the novel features of the present invention, if each spheroid was stained and analysed with confocal microscopy each one would have to be stained and imaged individually, and would be limited to 4 total types of stain because of limited channels. If conventional methods of sectioning were used, then 32 separate spheroids would have to be dehydrated, embedded, sectioned, and stained. With the system and methods described herein, all 32 spheroids were able to be sectioned from the microarray and because there were many slides, all 32 spheroids were able to be stained with H&E for cell nuclei and cell/particle organization, Masson's trichrome for extracellular matrix, 2 markers of adipogenesis, 2 markers of osteogenesis, 3 markers of chondrogenesis. This was all completed very efficiently.

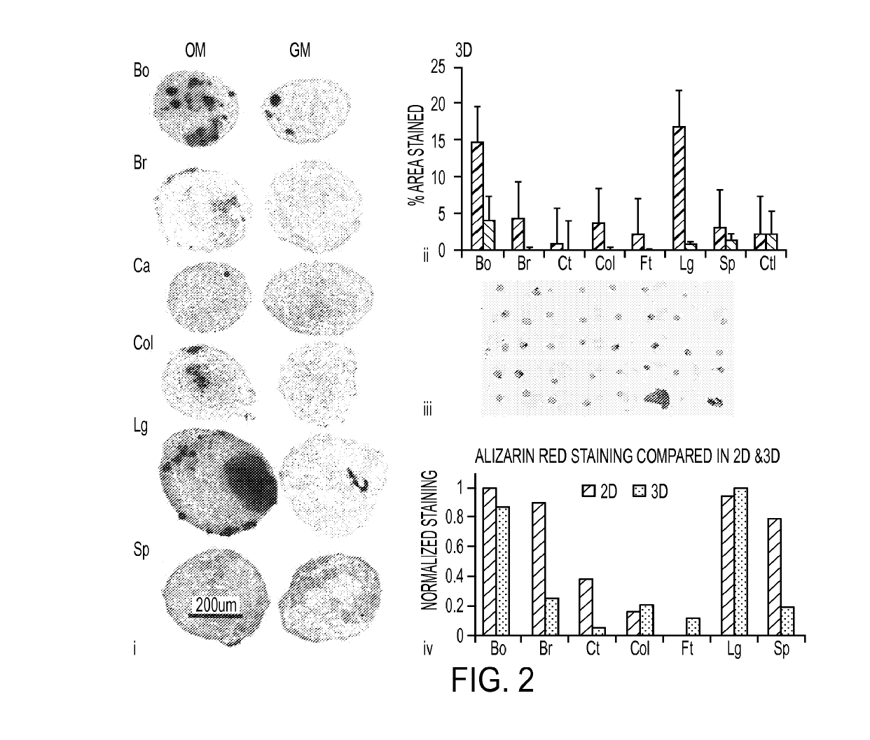

In these experiments, adipose derived stem cells were cultured with particles at a ratio of 850,000 cells/ml to 0.6 mg/ml of particles. 20 uL of each suspension was mixed and a 40 uL hanging drop was used to form a spheroid. These were cultured in basal media for 6 days and basal or osteogenic media for an additional 11 days, and then stained as described above. Alizarin red staining (stained calcified matrix production which is indicator of osteogenic differentiation) was quantified as shown in FIG. 2.

### Example 2

### Method of Preparing a Microarray of Cell Spheroids Using a Single Piece Micromold.

While certain of the above-exemplified methods for preparing a microarray of cell spheroids involved use of a plastic mold comprising through-holes that was pressed up against a PDMS backing, it was additionally contemplated that a single piece mold could be used in the methods of the invention. A single piece mold comprising an array of wells was therefore synthesized and employed.

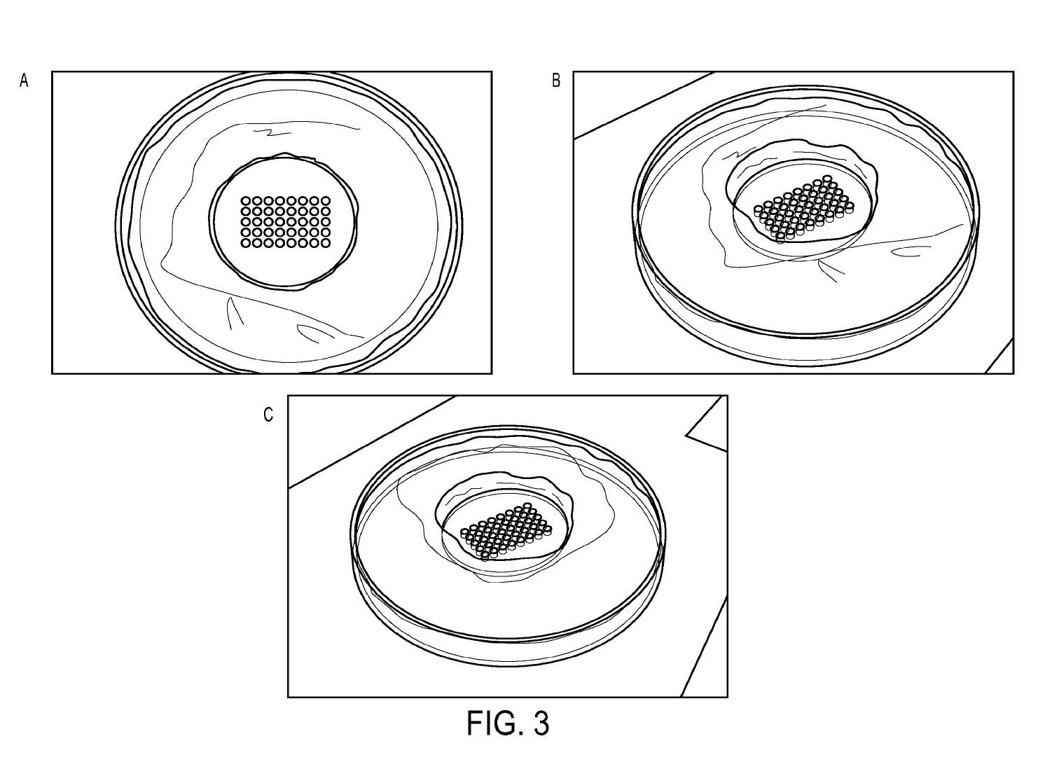

As shown in FIGS. 3A to 3C, a single piece micromold having an array of wells was synthesized from polydimethylsiloxane (PDMS) and was used in the methods of the invention. Testing of the single piece micromold identified it to function as well as the two piece micromold described above (data not shown).

### INCORPORATION BY REFERENCE

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

What is claimed is:

1. A method for preparing a microarray of cell spheroids comprising:
culturing a plurality of cell spheroids in at least one array plate comprising a top surface and a bottom surface and a plurality of holes therein, and configured to accommodate a plurality of hanging drops, wherein the hanging drops comprise one or more spheroids;
preparing a micromold having an array of wells;
transferring the cell spheroids to the micromold wells; and
filling the micromold with agarose.

2. The method of claim 1, wherein the micromold is a single piece micromold.

3. The method of claim 1, wherein the micromold is comprised of plastic or silicone, optionally, polydimethylsiloxane (PDMS).

4. The method of claim 1, further comprising a step of placing a mounting block over the micromold before filling the micromold with agarose.

5. A method selected from the group consisting of:
A method of preparing a microarray of cell spheroids comprising:
culturing a plurality of cell spheroids in at least one array plate comprising a top surface and a bottom surface and a plurality of holes therein, and configured to accommodate a plurality of hanging drops, wherein the hanging drops comprise one or more spheroids;
preparing a micromold by pressing it against a hydrophobic surface;
transferring the cell spheroids to the micromold;
placing a mounting block over the micromold; and
filling the micromold with agarose;
A method of preparing a micromold of embedded spheroids for histology comprising:
culturing a plurality of cell spheroids in at least one array plate comprising a top surface and a bottom surface and a plurality of holes therein, and configured to accommodate a plurality of hanging drops, wherein the hanging drops comprise one or more spheroids;
preparing a micromold by pressing it against a hydrophobic surface;
transferring the cell spheroids to the micromold;
placing a mounting block over the micromold;
filling the micromold with agarose; and
embedding the micromold in paraffin or cryomount; and
A method of screening a library of agents comprising:
culturing a plurality of cell spheroids in at least one array plate comprising a top surface and a bottom surface and a plurality of holes therein, and configured to accommodate a plurality of hanging drops, wherein each hanging drop comprises one or more spheroids;
introducing an agent or a combination of agents into each hanging drop;
preparing a micromold having an array of through-holes or wells;
transferring the cell spheroids to the micromold;
placing a mounting block over the micromold;
filling the micromold with agarose; and
embedding the micromold in paraffin or cryomount.

6. (canceled)

7. The method of claim 1, wherein each drop hangs from a corresponding one of the plurality of said holes and extends beneath the hole, wherein the number of hanging drops that the array plate can accommodate is equal to or less than the number of holes in the at least one array plate.

8. The method of claim 1, further comprising embedding the micromold in paraffin or cryomount.

9. The method of claim 5, wherein the hydrophobic surface is a silicone substrate.

10. The method of claim 1, further comprising sectioning the micromold and transferring the sections to slides, optionally further comprising staining the slides.

11. (canceled)

12. The method of claim 1, wherein the cell spheroids are derived from healthy subjects or subjects with diseases selected from the group consisting of degenerative diseases, cancer diseases, autoimmune and/or inflammatory diseases, cardiovascular diseases and neurological disorders.

13. The method of claim 1, wherein the cell spheroids are derived from stem cells, and/or wherein the spheroid is used to model a disease or disorder, and/or wherein the cell spheroids are treated with an agent during culturing in the at least one array plate.

14-16. (canceled)

17. The method of claim 5, wherein the step of preparing the micromold comprises pressing the micromold against a hydrophobic surface.

18-21. (canceled)

22. The method of claim 5, further comprising staining the slides for a marker of interest, optionally wherein the marker is a protein.

23. (canceled)

24. The method of claim 5, wherein one or more separate hanging drop is treated with the same agent or with a different concentration of the same agent, or is treated with a different agent or a different concentration of the different agent, or is treated as a control.

25-26. (canceled)

27. The method of claim 5, wherein the agent is selected from one or more of the group consisting of: native or endogenous ligand or ligands, a combinatorial library of small molecules, hormones, antibodies, polysaccharides, anti-cancer agents, natural products, terrestrial products, marine natural products, a molecule that binds with high affinity to a biopolymer such as a protein, a nucleic acid, and a polysaccharide, a purified or isolated biological molecule such as a protein, a nucleic acid, a silencing RNA (siRNA), a micro RNA (miRNA), and a short hairpin RNA (shRNA).

28. The method of claim 22, wherein detection of the marker indicates activity of the agent, or wherein absence of the marker indicates activity of the agent.

29. (canceled)

30. The method of claim 1, wherein the method is an in vitro method.

31. A composition selected from the group consisting of:
A micromold for embedding spheroids comprising a plurality of cell spheroids and a mounting block, wherein the micromold is filled with agarose; and
An agarose-embedded array comprising spheroids.

32-36. (canceled)

37. The agarose-embedded array of claim 31, wherein multiple array elements comprise one or more spheroids, or wherein each array element comprises one or more spheroids.

38. (canceled)

39. A method for comparing the staining intensities of different spheroids without normalizing to an external value, the method comprising staining the agarose-embedded array of claim 31, imaging the array on a single slide to obtain staining intensity values of different spheroids of the array, and directly comparing the staining intensity values of the different spheroids of the array.
